(19) United States
(12) Patent Application Publication
(10) Pub. No.: US 2022/0378777 A1
(43) Pub. Date: Dec. 1, 2022

(54) COMPOSITIONS AND METHODS FOR TREATING ALCOHOL USE DISORDER OR A RELATED CONDITION THEREOF

(71) Applicant: THE TRUSTEES OF INDIANA UNIVERSITY, Bloomington, IN (US)

(72) Inventor: Zachary A. Rodd, Indianapolis, IN (US)

(21) Appl. No.: 17/771,985

(22) PCT Filed: Oct. 30, 2020
(86) PCT No.: PCT/US2020/058212

§ 371 Date: Apr. 26, 2022

Related U.S. Application Data
(60) Provisional application No. 62/928,401, filed on Oct. 31, 2019.

(51) Int. Cl. A61K 31/4725; A61K 31/135; A61K 31/4375; A61P 25/32

(52) U.S. Cl. CPC A61K 31/4725; A61K 31/135; A61K 31/4375; A61P 25/32

(57) ABSTRACT

Various aspects and embodiments disclosed herein relate generally to treating an alcohol use disorder or neuroadaptations. Embodiments include compositions and methods for modelling, treatment, reducing resistance to the treatment, prevention, and diagnosis of a condition/disease associated with alcohol use disorders, or a related clinical condition thereof. Other embodiments include methods and compositions for reducing the effects of adolescent alcohol drinking.

### CROSS-REFERENCE TO RELATED APPLICATIONS

This application claims the benefit of U.S. Provisional Application No. 62/928,401, filed Oct. 31, 2019, the entire disclosure of which is hereby expressly incorporated by reference herein.

### STATEMENT OF GOVERNMENTAL RIGHTS

This invention was made with government support under AA024612 awarded by National Institutes of Health. The government has certain rights in the invention.

### FIELD

Various aspects and embodiments disclosed herein relate generally to treating an alcohol use disorder or neuroadaptations produced by alcohol drinking. Embodiments include compositions and methods for modelling, treatment, reducing resistance to the treatment, prevention, and diagnosis of a condition/disease associated with alcohol use disorders, or a related clinical condition thereof. Other embodiments include methods and compositions for reducing the effects of adolescent alcohol drinking.

### BACKGROUND

Binge drinking or drinking intoxicating doses of alcohol that elevates blood alcohol levels to greater than 0.08 g/dL may develop alcohol use disorders. Adolescents respond differently to alcohol than adults, and further, the adolescent brain appears to be more susceptible to the effects of binge alcohol consumption than the adult brain. Neuroadaptations produced by adolescent alcohol drinking are thought to be the basis of an increase in alcoholism in adults. Alcohol consumption during adolescence is associated with a number of deleterious consequences. Age of first alcohol drink and the propensity to have binge alcohol drinking episodes during adolescence is associated with heavier drinking bouts, arrests for driving with ability impaired, and an increased rate of alcohol dependence during adulthood.

Epidemiological data suggests that early initiation of alcohol consumption is associated with a greater risk of developing an alcohol use disorder (AUD) during adulthood. Binge-like alcohol consumption during adolescence associates with several deleterious consequences during adulthood including an increased risk for developing AUD and other addictions. For example, adolescent alcohol drinking has been linked to increased adulthood use of opioids, cannabis, and other drugs of abuse. Specifically, for nicotine, adolescent binge drinking enhances the likelihood of smoking during adolescence by 88% as well as during adulthood. In contrast, individuals who do not engage in binge drinking have lower rates of smoking during adolescence and adulthood.

Despite numerous studies examining the consequence of adolescent alcohol consumption on alterations in the adult brain, there have been few studying the relationship between binge-like alcohol consumption during adolescence and the consequential effects observed during adulthood. Thus, a need exists for the development of a therapeutic to effectively treat harmful neuroadaptations or a similar condition thereof that result from binge-like alcohol consumption during adolescence.

### SUMMARY

Embodiments of the instant application relates to treating an alcohol use disorder or neuroadaptations produced by adolescent alcohol drinking. Embodiments include compositions and methods for modelling, treatment, reducing resistance to the treatment, prevention, and diagnosis of a condition/disease associated with alcohol use disorders, or a related clinical condition thereof. Yet other embodiments include methods and compositions for reducing the likelihood or potential effects of adolescent alcohol drinking.

A first embodiment includes a method of treating an alcohol use disorder or a related condition thereof, comprising: administering to a subject at least one therapeutically effective dose of at least one agent comprising at least one negative modulator of alpha-7 nicotinic receptor.

A second embodiment includes the method according to the first embodiment, wherein the at least one negative modulator comprises at least one of: norketamine ((2-amino-2-(2-chlorophenyl)cyclohexan-1-one), dehydronorketamine (5,6-dehydronorketamine), hydroxynorketamine ((2-amino-2-(2-chlorophenyl)-6-hydroxycyclohexan-1-one), SB-27701-A ((N-{trans-4-[2-(6-cyano-3,4-dihydroisoquinolin-2(1H)-yl)ethyl]cyclohexyl}quinoline-4-carboxamide), butaclamol ((3S,4aS,13bS)-3-(2-methyl-2-propanyl)-2,3,4,4a,8,9,13b,14-octahydro-1Hbenzo[6,7]cyclohepta[1,2,3-de]pyrido[2,1-a]isoquinolin-3-ol), DB04763 ((2,4,6-trimethyl-1-[2-(4-sulfamoylphenyl)ethyl]pyridin-1-ium), DB08122 ((N-methyl-4-({[(3Z)-2-oxo-2,3-dihydro-1H-indol-3-ylidene]methyl}amino)benzene-1-sulfonamide), MD-354 (2-(3-chlorophenyl)-1-(diaminomethylidene)guanidine), HDMP ((1,2,3,3a,4,8b-hexahydro-2-benzyl-6-N,N-dimethylamino-1-methylindeno[1,2,-b]pyrrole), and perfloxacin, and/or analogs or metabolites thereof, or pharmaceutical salts thereof.

A third embodiment includes the method according to the first and the second embodiments, wherein the at least one negative modulator comprises at least one of: dehydronorketamine (5,6-dehydronorketamine), SB-27701-A ((N-{trans-4-[2-(6-cyano-3,4-dihydroisoquinolin-2(1H)-yl)ethyl]cyclohexyl}quinoline-4-carboxamide), and butaclamol ((3 S,4aS,13b S)-3-(2-methyl-2-propanyl)-2,3,4,4a, 8,9,13b,14-octahydro-1Hbenzo[6,7]cyclohepta[1,2,3-de]pyrido[2,1-a]isoquinolin-3-ol), and/or analogs or metabolites thereof, or pharmaceutical salts thereof.

A fourth embodiment includes the method according to any one of the first to the third embodiments, wherein the subject is diagnosed with or at risk of having an alcohol use disorder, a drug or alcohol addiction, an anxiety disorder, or a related condition thereof, and/or wherein the subject is a human or an animal, and/or wherein the subject is an adolescent, a young adult, or an adult.

A fifth embodiment includes the method according to any one of the first to the fourth embodiments, wherein the subject is diagnosed with or at risk of having or has increased likelihood of developing schizophrenia, Alzheimer's disease, Parkinson's disorder, other dementia-related illnesses, Type II diabetes, and autoimmune diseases, or a related condition thereof.

A sixth embodiment includes the method according to any one of the first to the fifth embodiments, wherein the subject is an adolescent or a young adult in the age between 9 to 25 years old, between 10-21 years old, between 10-19 years old, between 13-25 years old, between 13-21 years old, between 15-25 years old, between 15-21 years old, or any combination thereof.

A seventh embodiment includes the method according to any one of the first to the sixth embodiments, wherein the therapeutically effective dose of at least one agent comprises from about 0.01 mg/kg to about 1000 mg/kg, from about 0.01 mg/kg to about 500 mg/kg, from about 0.01 mg/kg to about 200 mg/kg, from about 0.01 mg/kg to about 100 mg/kg, from about 0.01 mg/kg to about 50 mg/kg, from about 0.01 mg/kg to about 10 mg/kg, from about 0.1 mg/kg to about 1000 mg/kg, from about 0.1 mg/kg to about 500 mg/kg, from about 0.1 mg/kg to about 200 mg/kg, from about 0.1 mg/kg to about 100 mg/kg, from about 0.1 mg/kg to about 50 mg/kg, from about 0.1 mg/kg to about 10 mg/kg, from about 1 mg/kg to about 1000 mg/kg, from about 1 mg to about 500 mg/kg, from about 1 mg/kg to about 200 mg/kg, from about 1 mg/kg to about 100 mg/kg, from about 1 mg/kg to about 50 mg/kg, from about 1 mg/kg to about 10 mg/kg, from about 10 mg/kg to about 1000 mg/kg, from about 10 mg/kg to about 500 mg/kg, from about 10 mg/kg to about 200 mg/kg, from about 10 mg/kg to about 100 mg/kg, from about 10 mg/kg to about 50 mg/kg, or any combination thereof, and/or wherein the therapeutically effective dose of at least one agent comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 60, 70, 80, 90, 100, 110, 120, 130, 140, 150, 160, 170, 180, 190, and/or 200 mg/kg, and/or wherein the therapeutically effective dose of at least one agent is administered orally, parenterally, rectally, and/or transdermally.

An eighth embodiment includes the method according to any one of the first to the seventh embodiments, further comprising the step of: identifying a subject who had experienced and/or exposed to binge/binge-like drinking.

A ninth embodiment includes the method according to any one of the first to the eighth embodiments, further comprising the step of: administering to the subject the therapeutically effective dose of at least one agent at least once prior to, during, or subsequent to alcohol consumption.

A tenth embodiment includes the method of any one of the first to the ninth embodiments, wherein the therapeutically effective dose of at least one agent is administered to the subject at least once within about 1, 2, 3, 4, 5, 6, 7, 8, 9, and/or 10 hours prior to or subsequent to alcohol consumption.

An eleventh embodiment includes a method of reducing alcohol consumption, comprising: administering to an adolescent subject at least one therapeutically effective dose of at least one agent comprising at least one negative modulator of alpha-7 nicotinic receptor.

A twelfth embodiment includes the method according to the eleventh embodiment, wherein the at least one negative modulator comprises at least one of: norketamine ((2-amino-2-(2-chlorophenyl)cyclohexan-1-one), dehydronorketamine (5,6-dehydronorketamine), hydroxynorketamine ((2-amino-2-(2-chlorophenyl)-6-hydroxycyclohexan-1-one), SB-27701-A ((N-{trans-4-[2-(6-cyano-3,4-dihydroisoquinolin-2(1H)-yl)ethyl]cyclohexyl}quinoline-4-carboxamide), butaclamol ((3S,4aS,13bS)-3-(2-methyl-2-propanyl)-2,3,4,4a,8,9,13b,14-octahydro-1Hbenzo[6,7]cyclohepta[1,2,3-de]pyrido[2,1-a]isoquinolin-3-ol), DB04763 ((2,4,6-trimethyl-1-[2-(4-sulfamoylphenyl)ethyl]pyridin-1-ium), DB08122 ((N-methyl-4-({[(3Z)-2-oxo-2,3-dihydro-1H-indol-3-ylidene]methyl}amino)benzene-1-sulfonamide), MD-354 (2-(3-chlorophenyl)-1-(diaminomethylidene)guanidine), HDMP ((1,2,3,3a,4,8b-hexahydro-2-benzyl-6-N,N-dimethylamino-1-methylindeno[1,2,-b]pyrrole), and perfloxacin, and/or analogs or metabolites thereof, or pharmaceutical salts thereof.

A thirteenth embodiment includes the method according to the eleventh and the twelfth embodiments, wherein the at least one negative modulator comprises at least one of: dehydronorketamine (5,6-dehydronorketamine), SB-27701-A ((N-{trans-4-[2-(6-cyano-3,4-dihydroisoquinolin-2(1H)-yl)ethyl]cyclohexyl}quinoline-4-carboxamide), and butaclamol ((3 S,4aS,13b S)-3-(2-methyl-2-propanyl)-2,3,4,4a,8,9,13b,14-octahydro-1Hbenzo[6,7]cyclohepta[1,2,3-de]pyrido[2,1-a]isoquinolin-3-ol), and/or analogs or metabolites thereof, or pharmaceutical salts thereof.

A fourteenth embodiment includes the method according to any one of the eleventh to the thirteenth embodiments, wherein the adolescent subject has been exposed to binge or binge-like alcohol consumption.

A fifteenth embodiment includes the method according to any one of the eleventh to the fourteenth embodiments, wherein the adolescent subject is diagnosed with or at risk of having or has increased likelihood of developing alcohol use disorder, a drug or alcohol addiction, an anxiety disorder, schizophrenia, Alzheimer's disease, Parkinson's disorder, other dementia-related illnesses, Type II diabetes, and autoimmune diseases, or a related condition thereof when the adolescent subject becomes an adult, and/or wherein the adolescent subject experiences reduced likelihood of developing alcohol use disorder, a drug or alcohol addiction, an anxiety disorder, schizophrenia, Alzheimer's disease, Parkinson's disorder, other dementia-related illnesses, Type II diabetes, and autoimmune diseases, or a related condition thereof when the adolescent subject becomes an adult.

A sixteenth embodiment includes the method according to any one of the eleventh to the fifteenth embodiments, wherein the adolescent subject is in the age between 9 to 25 years old, between 10 to 25 years old, between 11 to 25 years old, between 12 to 25 years old, between 13 to 25 years old, between 14 to 25 years old, between 15 to 25 years old, between 16 to 25 years old, between 17 to 25 years old, between 18 to 25 years old, between 19 to 25 years old, between 9 to 20 years old, between 10 to 20 years old, between 11 to 20 years old, between 12 to 20 years old, between 13 to 20 years old, between 14 to 20 years old, between 15 to 20 years old, between 16 to 20 years old, between 17 to 20 years old, between 18 to 20 years old, between 19 to 20 years old, or any combination thereof, and/or about 9, 10, 11, 12, 13, 14, 15, 16, 17, 18, 19. 20, 21, 22, 23, 24, and/or 25 years old.

A seventeenth embodiment includes the method according to any one of the eleventh to the sixteenth embodiments, wherein the therapeutically effective dose of at least one agent comprises from about 0.01 mg/kg to about 1000 mg/kg, from about 0.01 mg/kg to about 500 mg/kg, from about 0.01 mg/kg to about 200 mg/kg, from about 0.01 mg/kg to about 100 mg/kg, from about 0.01 mg/kg to about 50 mg/kg, from about 0.01 mg/kg to about 10 mg/kg, from about 0.1 mg/kg to about 1000 mg/kg, from about 0.1 mg/kg to about 500 mg/kg, from about 0.1 mg/kg to about 200 mg/kg, from about 0.1 mg/kg to about 100 mg/kg, from about 0.1 mg/kg to about 50 mg/kg, from about 0.1 mg/kg to about 10 mg/kg, from about 1 mg/kg to about 1000 mg/kg, from about 1 mg to about 500 mg/kg, from about 1 mg/kg to about 200 mg/kg, from about 1 mg/kg to about 100 mg/kg, from about 1 mg/kg to about 50 mg/kg, from about 1 mg/kg to about 10 mg/kg, from about 10 mg/kg to about 1000 mg/kg, from about 10 mg/kg to about 500 mg/kg, from about 10 mg/kg to about 200 mg/kg, from about 10 mg/kg to about 100 mg/kg, from about 10 mg/kg to about 50 mg/kg, or any combination thereof, and/or wherein the therapeutically effective dose of at least one agent comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 60, 70, 80, 90, 100, 110, 120, 130, 140, 150, 160, 170, 180, 190, and/or 200 mg/kg, and/or wherein the therapeutically effective dose of at least one agent is administered orally, parenterally, rectally, and/or transdermally.

An eighteenth embodiment includes the method according to any one of the eleventh to the seventeenth embodiments, further comprising the step of: administering to the adolescent subject the therapeutically effective dose of at least one agent at least once prior to, during, or subsequent to alcohol consumption.

A nineteenth embodiment includes the method according to any one of the eleventh to the eighteenth embodiments, further comprising the step of: administering to the adolescent subject the therapeutically effective dose of at least one agent at least once prior to binge or binge-like drinking/alcohol consumption.

A twentieth embodiment includes the method according to any one of the eleventh to the nineteenth embodiments, wherein the therapeutically effective dose of at least one agent is administered to the subject at least once within about 1, 2, 3, 4, 5, 6, 7, 8, 9, and/or 10 hours prior to or subsequent to alcohol consumption.

### BRIEF DESCRIPTION OF THE DRAWINGS

The following drawings form part of the present specification and are included to further demonstrate certain embodiments. Some embodiments may be better understood by reference to one or more of these drawings alone or in combination with the detailed description of specific embodiments presented.

FIG. 1 depicts the methods used to determine the effects of exposure to α7 NAchR agonists during adolescence on adult alcohol consumption (top panel), and the ability of an α7 NAchR negative allosteric modulator to prevent the effects of adolescence binge alcohol exposure to enhance adult alcohol consumption (bottom panel).

FIG. 2 depicts the average alcohol intake in female (top) and male (bottom) rats administered AR-R17779 during adolescence on adult alcohol consumption. * indicates 10 mg/kg AR-R17779>3 mg/kg and saline. # indicates 3 mg/kg>saline.

FIG. 3 depicts the mean (+SEM) for female (top) and male (bottom) alcohol-preferring (P) rats during relapse EtOH drinking. * indicates EtOH consumption exceeds baseline intake. + indicates that rats administered 10 mg/kg AR-R177779 during adolescence consumed more alcohol than saline controls.

FIG. 4 depicts the mean (+SEM) for Wistar rats self-administering beer during adulthood following AR treatment during adolescence. * indicates 3 and 10 AR>saline. +10>3>saline.

FIG. 5 depicts the average alcohol intake in male (top) and female (bottom) rats administered DHNK 2 hours prior to AIE or CON treatment during adolescence. * indicates AIE-Saline rats consumed more alcohol than all other groups.

FIG. 6 Depicts the mean (+SEM) for female (top) and male (bottom) alcohol-preferring (P) rats during relapse EtOH drinking. * indicates EtOH consumption exceeds baseline intake. + indicates that rat pretreated with saline prior to AIE exposure (AIE-Saline) consumed more alcohol than any other group.

FIG. 7 depicts the average alcohol intake in female P rats administered the α7 NAM SB-277011-A 2 hr before ABAE treatment during adolescence on the adult consumption of EtOH. * indicates EtOH consumption in water-ABAE rats >1 mg and 5 mg/kg SB rats. + indicates 1 mg/kg>5 mg/kg SB rats. # indicates water-ABAE and 1 mg/kg SB>5 mg/kg SB rats.

FIG. 8 depicts the mean (+SEM) for female alcohol-preferring (P) rats during relapse EtOH drinking. * indicates EtOH consumption exceeds baseline intake. + indicates that rat pretreated with saline prior to ABAE exposure (water and 1 mg/kg SB) consumed more alcohol than corresponding CON group.

### DEFINITIONS

“About” refers to a range of values plus or minus 10 percent, e.g. about 1.0 encompasses values from 0.9 to 1.1.

“Alcohol use disorder or a related condition thereof” refers to a chronic disease characterized by uncontrolled drinking and preoccupation with alcohol. It may include, but is not limited to, alcoholism, alcohol abuse, a drug or alcohol addiction, and an anxiety disorder, each of which can develop or lead to any one of schizophrenia, Alzheimer's disease, Parkinson's disorder, other dementia-related illnesses, Type II diabetes, and autoimmune diseases.

“Binge drinking” or “binge-like drinking” refers to a pattern of drinking that brings a person's blood alcohol concentration (BAC) to 0.08 g/dl or above. This typically happens when men consume 5 or more drinks or women consume 4 or more drinks in about 2 hours.

“One drink” or “one alcohol drink” refers to one standard drink as defined by the National Institute on Alcohol Abuse and Alcoholism and includes any one of the following: (1) 12 ounces (355 milliliters) of regular beer (about 5 percent alcohol); (2) 8 to 9 ounces (237 to 266 milliliters) of malt liquor (about 7 percent alcohol); (3) 5 ounces (148 milliliters) of unfortified wine (about 12 percent alcohol); and (4) 1.5 ounces (44 milliliters) of 80-proof hard liquor (about 40 percent alcohol).

“Subject” refers to a mammal or a human.

“Therapeutically effective dose” or “therapeutically effective amount” refers to a dose or amount that provides effective treatment of a disease or disorder in a subject. A therapeutically effective dose can vary from compound to compound, from cell to cell, and from subject to subject, and can depend upon factors such as the condition of the subject, the route of delivery, the disease and/or symptoms of the disease, severity of the disease and/or symptoms of the disease or disorder, the age, weight, and/or health of the subject to be treated, and the judgment of the prescribing physician.

“Treat,” “treating” or “treatment” of any disease refers to reversing, alleviating, arresting, or ameliorating a disease or at least one of the clinical symptoms of a disease, reducing the risk of acquiring a disease or at least one of the clinical symptoms of a disease, inhibiting the progress of a disease or at least one of the clinical symptoms of the disease or reducing the risk of developing a disease or at least one of the clinical symptoms of a disease. “Treat,” “treating” or “treatment” also refers to inhibiting the disease, either physically, (e.g., stabilization of a discernible symptom), physiologically, (e.g., stabilization of a physical parameter), or both, and to inhibiting at least one physical parameter that can or cannot be discernible to the subject. In certain embodiments, “treat,” “treating” or “treatment” refers to delaying the onset of the disease or at least one or more symptoms thereof in a subject which can be exposed to or predisposed to a disease even though that subject does not yet experience or display symptoms of the disease.

### DETAILED DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

The adolescent brain is in neuro-developmental flux and is characterized by rapid growth, reorganization, and pruning of neurons throughout adolescence. External factors can impact the normal developmental procedures and result in maladaptive consequences. Therefore, adolescence is also a unique time period in which interventions can be performed to prevent propagating consequences within the human brain. The focus of pharmacological treatment during adolescence should be prevention.

In the USA, 12th graders 58% report the use of alcohol within the past year and 28% of which engaged in binge drinking within the previous 2 weeks (i.e. >5 consecutive drinks per drinking episode). Binge drinking is defined by the National Institute on Alcohol Abuse and Alcoholism as 4+/5+ drinkers for women/men per occasion (or a blood alcohol level of 0.08 g/dL (CDC, 2013). Binge drinking is exaggerated in US young adults (age 18-24) since the average binge episode consisted of 9.5 drinks/occasion. A recent trend in adolescent/young adult drinking is that the starting point of the incidents of binge drinking has reduced and a sharper increase in the overall rate of binge drinking during the transition from late adolescent/young adulthood into adulthood. In addition, high-intensity and extreme-intensity adolescents has recently emerged from assessment of adolescent drinking in the USA.

Epidemiological studies indicate 1.3 to 1.6 times increased lifetime rate of AUD in individuals who initiate alcohol use before the age of 15. The negative effects of adolescent alcohol consumption on adult alcohol dependence are compounded in individuals with a family history of alcoholism. Specifically, a family history of alcoholism interacts with the age of onset of drinking to significantly increase the rate of adult AUD. A family history of alcoholism also significantly increased alteration in white matter integrity (fractional anisotropy) observed following adolescent binge alcohol consumption. Young adults consuming high-intensity levels of alcohol (8.6 g/day) displayed an increase in alcohol consumption during later adulthood and higher levels of AUD. Specifically, the total consumed dose of alcohol during young adulthood increased future risk of AUD, but a pattern of heavy episodic drinking (high-intensity drinking) significantly increase later risk of AUD and cirrhosis. Preventing early adolescent alcohol consumption has a protective effect on drinking patterns during late adolescent and adulthood.

Preclinically, adolescent alcohol exposure enhances adult alcohol consumption in rodents. Similarly, in humans, equivalent exposure to alcohol in adolescence results in unique alterations in alcohol consumption (enhancement) while comparable adult alcohol exposure does not result in similar alterations in self-administration behaviors. Adolescent alcohol exposure produces; 1) hyperdopaminergic system, 2) reduction in choline acetyltransferase (ChAT), 3) alterations in neurohormones, 4) alterations in epigenetic factors, 5) an increase in the predisposition for other disorders, and 6) alterations in numerous behaviors (increased anxiety, increased drug use, etc). The α7 nicotinic acetylcholine receptors (NAchR). Alcohol primarily acts upon cys-loop receptors in the brain; α7 NAchR, serotonin 3 (5HT₃), GABA_{A}, glycine, and some glutamate receptors. The α7 NAchR regulates all of the observed adult consequences of adolescent alcohol consumption.

Neuroprotection produced by an alpha-7 nicotinic receptor negative allosteric modulator prevents the dysregulation in the adult alpha-7 nicotinic receptor and reduces the likelihood of psoriasis, rheumatoid arthritis, and systemic lupus erythematosus in adulthood.

The neuroprotection by treatment of an alpha-7 nicotinic receptor negative allosteric modulator to reduce the deleterious effects of adolescent alcohol consumption are potentially two-fold. Treatment with alpha-7 nicotinic receptor negative allosteric modulator can reduce the harmful effects of alcohol on the developing adolescent/young adult brain. This action would reduce the direct harm that adolescent alcohol consumption would cause within the brain that produces disorders during adulthood. Treatment with alpha-7 nicotinic receptor negative allosteric modulator also can prevent subsequent behaviors (e.g. adult drug use) that increases the risk factor of developing an adult disorder. The alpha-7 nicotinic receptor negative allosteric modulator may be administered to an adolescent where the neuroprotective effect is against an adult brain pathology. The alpha-7 nicotinic receptor negative allosteric modulator may be administered within about four hours before to about four hours after the subject consumes alcohol.

The negative allosteric modulators of the invention, or salts thereof, may be prepared by a variety of procedures known to one of ordinary skill in the art, some of which are illustrated in the schemes, preparations, and examples below. One of ordinary skill in the art recognizes that the specific synthetic steps for each of the routes described may be combined in different ways, or in conjunction with steps from different schemes, to prepare compounds of the invention, or salts thereof. The products of each step in the schemes below can be recovered by conventional methods well known in the art, including extraction, evaporation, precipitation, chromatography, filtration, trituration, and crystallization. In the schemes below, all substituents unless otherwise indicated are as previously defined. The reagents and starting materials are readily available to one of ordinary skill in the art. Others may be made by standard techniques of organic and heterocyclic chemistry which are analogous to the syntheses of known structurally similar compounds and the procedures subsequently described, including any novel procedures. Without limiting the scope of the invention, the following schemes, preparations, and examples are provided to further illustrate the invention. In addition, one of ordinary skill in the art appreciates that the compounds may be prepared by using starting material with the corresponding stereochemical configuration which can be prepared by one of skill in the art.

A negative allosteric modulator (NAM) is not an antagonist. A negative allosteric modulator acts at an allosteric site on a receptor to reduce activity induced by ligands. A negative allosteric modulator does not reduce the activity of a receptor; it restricts the increase or decrease activity of a receptor produced by ligands. The use of a α7 nicotinic receptor negative allosteric modulator is beneficial to use in adolescent subjects because it does not alter normal activity of a receptor. Inhibition of the α7 nicotinic receptor, or reduction in the genetic expression of the α7 nicotinic receptor, during adolescence impairs cognitive performance during adulthood. In contrast, no published findings indicate that a α7 nicotinic receptor negative allosteric modulator produces that negative side effect.

The present disclosure contemplates all individual enantiomers and diastereomers, as well as mixtures of the enantiomers of the alpha-7 nicotinic receptor negative allosteric modulator compounds, including racemates. Individual isomers, enantiomers, and diastereomers may be separated or resolved by one of ordinary skill in the art at any convenient point in the synthesis of compounds of the invention, by methods such as selective crystallization techniques or chiral chromatography (see, e.g., Jacques et al. Enantiomers, Racemates, and Resolutions, John Wiley and Sons, Inc., 1981, and Eliel and Wilen, Stereochemistry of Organic Compounds, Wiley-Interscience, 1994). The alpha-7 nicotinic receptor negative allosteric modulator compounds are preferably formulated as pharmaceutical compositions administered by any route which makes the compound bioavailable, including oral and transdermal routes. Most preferably, such compositions are for oral administration. Such pharmaceutical compositions and processes for preparing same are well known in the art (See, e.g., Remington: The Science and Practice of Pharmacy, L. V. Allen, Editor, 22^{nd} Edition, Pharmaceutical Press, 2012).

### Pharmaceutical Compositions

Pharmaceutical compositions provided by the present disclosure can comprise a therapeutically effective amount of one or more compositions disclosed herein, together with a suitable amount of one or more pharmaceutically acceptable vehicles to provide a composition for proper administration to a subject. Suitable pharmaceutical vehicles are described in the art.

Pharmaceutical compositions of the present disclosure suitable for oral administration can be presented as discrete units, such as a capsule, cachet, tablet, or lozenge, each containing a predetermined amount of the active ingredient; as a powder or granules; as a solution or a suspension in an aqueous liquid or non-aqueous liquid such as a syrup, elixir or a draught, or as an oil-in-water liquid emulsion or a water-in-oil liquid emulsion. The composition can also be presented as a bolus, electuary or paste. A tablet can be made by compressing or moulding the active ingredient with the pharmaceutically acceptable carrier. Compressed tablets can be prepared by compressing in a suitable machine the active ingredient in a free-flowing form, such as a powder or granules, in admixture with, for example, a binding agent, an inert diluent, a lubricating agent, a disintegrating and/or a surface-active agent. Moulded tablets can be prepared by moulding in a suitable machine a mixture of the powdered active ingredient moistened with an inert liquid diluent. The tablets can optionally be coated or scored and can be formulated to provide slow or controlled release of the active ingredient.

Pharmaceutical compositions of the present disclosure suitable for parenteral administration include aqueous and non-aqueous sterile injection solutions, and can also include an antioxidant, buffer, a bacteriostat and a solution which renders the composition isotonic with the blood of the recipient, and aqueous and non-aqueous sterile suspensions which can contain, for example, a suspending agent and a thickening agent. The formulations can be presented in a single unit-dose or multi-dose containers and can be stored in a lyophilized condition requiring the addition of a sterile liquid carrier prior to use.

In some embodiments, the composition can contain pharmaceutically acceptable lubricant(s). The pharmaceutically acceptable lubricant(s) prevent the components of the pharmaceutical composition from clumping together and from sticking to the pellet press that generates the disclosed compositions. The lubricant(s) also ensure that the formation of the pellet, as well as its ejection from the pellet press, occurs with low friction between the composition and the wall of the die press. In some embodiments, the lubricant(s) are added to a pharmaceutical composition to improve processing characteristics, for example to help increase the flexibility of the compositions, thereby reducing breakage.

The type of lubricant that can be used in the disclosed pharmaceutical compositions can vary. In some embodiments, the pharmaceutically acceptable lubricant is selected from talc, silica, vegetable stearin, magnesium stearate, stearic acid, calcium stearate, glyceryl behenate, glyceryl monostearate, glyceryl palmitostearate, mineral oil, polyethylene glycol, sodium stearyl fumarate, sodium lauryl sulfate, vegetable oil, zinc stearate, and combinations thereof. In some embodiments, the pharmaceutically acceptable lubricant is stearic acid.

The type of vehicles that can be used in the disclosed pharmaceutical compositions can vary. In some embodiments, the pharmaceutically acceptable vehicles are selected from binders, fillers and combinations thereof. In some embodiments, the pharmaceutically acceptable vehicle is selected from ascorbic acid, polyvinylpyrrolidone, polyvinylpyrrolidone K-30 (povidone K-30), glyceryl monostearate (GMS) or glyceryl monostearate salts, glyceryl behenate, glyceryl palmitostearate, hydroxypropyl cellulose, hydroxypropyl methylcellulose, methylcellulose, hydroxyethyl cellulose, sugars, dextran, cornstarch, dibasic calcium phosphate, dibasic calcium phosphate dihydrate, calcium sulfate, dicalcium phosphate, tricalcium phosphate, lactose, cellulose including microcrystalline cellulose, mannitol, sodium chloride, dry starch, pregelatinized starch, compressible sugar, mannitol, lactose monohydrate, starch, dibasic calcium phosphate dihydrate, calcium sulfate, dicalcium phosphate, tricalcium phosphate, powdered cellulose, microcrystalline cellulose, lactose, glucose, fructose, sucrose, mannose, dextrose, galactose, the corresponding sugar alcohols and other sugar alcohols, such as mannitol, sorbitol, xylitol, and combinations of any of the foregoing. In some embodiments, the pharmaceutically acceptable vehicle is polyvinylpyrrolidone K-30, also known as povidone K-30. In some embodiments, the pharmaceutically acceptable vehicle is polyvinylpyrrolidone K-30, also known as povidone K-30, having an average molecular weight of MW of 40,000 (CAS 9003-39-8). In some embodiments, the pharmaceutically acceptable vehicle is selected from glyceryl monostearate (GMS) or glyceryl monostearate salts, glyceryl behenate and glyceryl palmitostearate. In some embodiments, the pharmaceutically acceptable vehicle is glyceryl monostearate (GMS) or glyceryl monostearate salts. In some embodiments, the pharmaceutically acceptable vehicle is glyceryl behenate. In some embodiments, the pharmaceutically acceptable vehicle is glyceryl palmitostearate.

In some embodiments, the antioxidants prevent oxidation of the other components of the disclosed compositions. Oxidation can occur, for example, during sterilization where free radicals are generated. Addition of the antioxidants, or free radical scavengers, significantly reduces oxidation and makes the composition more pharmaceutically acceptable for use in subjects.

The type of antioxidants that can be used in the disclosed pharmaceutical compositions can vary. In some embodiments, the antioxidant is selected from methyl paraben and salts thereof, propyl paraben and salts thereof, vitamin E, vitamin E TPGS, propyl gallate, sulfites, ascorbic acid (aka L-ascorbic acid, also including the L-enantiomer of ascorbic acid, vitamin C), sodium benzoate, citric acid, cyclodextrins, peroxide scavengers, benzoic acid, ethylenediaminetetraacetic acid (EDTA) and salts thereof, chain terminators (e.g., thiols and phenols), butylated hydroxytoluene (BHT), butylated hydroxyanisole (BHA), and combinations thereof.

### Uses

The methods and compositions disclosed herein can be used to treat subjects suffering from diseases, disorders, conditions, and symptoms for which modulators of alpha-7 nicotinic receptor are known to provide or are later found to provide therapeutic benefit.

In some embodiments, methods disclosed herein include a method of treating a clinical condition, comprising administering to a subject at least one therapeutically effective dose of any of the compositions disclosed herein. The subject can be diagnosed with or at risk of having a clinical condition selected from and/or comprising wherein the subject is diagnosed with an alcohol use disorder, a drug or alcohol addiction, an anxiety disorder, neuroadaptations, or a related condition thereof, and/or the subject can be diagnosed with or at risk of having of developing schizophrenia, Alzheimer's disease, Parkinson's disorder, other dementia-related illnesses, Type II diabetes, and autoimmune diseases.

In certain embodiments, the methods disclosed herein further comprise administering to the subject at least one additional therapeutically effective dose of any of the compositions disclosed herein. In some embodiments, the at least one therapeutically effective dose of any of the compositions disclosed herein can be administered orally, parenterally, intravenously, by inhalation and/or transdermally.

Yet other embodiments can include methods for reducing a side effect of a therapeutic regime, comprising administering to a subject at least one therapeutically effective dose of at least one agent that inhibits or negatively modulates the activity of alpha-7 nicotinic receptor in a subject; wherein the subject has received at least one therapeutic regime comprising counseling, such as behavioral therapy, medications that reduce the desire to drink, medical detoxification to stop drinking safely and wherein the subject experiences at least one side effect derived from the therapeutic regime. Consistent with these embodiments, side effects can include, but are not limited to, drug-resistance and relapse.

In accordance with these embodiments, the at least one therapeutically effective dose of at least one agent that or negatively modulates the activity of alpha-7 nicotinic receptor comprises at least one of norketamine ((2-amino-2-(2-chlorophenyl)cyclohexan-1-one), dehydronorketamine (5,6-dehydronorketamine), hydroxynorketamine ((2-amino-2-(2-chlorophenyl)-6-hydroxycyclohexan-1-one), SB-27701-A 4N-{trans-4-[2-(6-cyano-3,4-dihydroisoquinolin-2(1H)-yl)ethyl]cyclohexyl}quinoline-4-carboxamide), butaclamol ((3 S,4aS,13b S)-3-(2-methyl-2-propanyl)-2,3,4,4a,8,9,13b,14-octahydro-1Hbenzo[6,7]cyclohepta[1,2,3-de]pyrido[2,1-a]isoquinolin-3-ol), DB04763 ((2,4,6-trimethyl-1-[2-(4-sulfamoylphenyl)ethyl]pyridin-1-ium), DB08122 ((N-methyl-4-({[(3Z)-2-oxo-2,3-dihydro-1H-indol-3-ylidene]methyl}amino)benzene-1-sulfonamide), MD-354 (2-(3-chlorophenyl)-1-(diaminomethylidene)guanidine), HDMP ((1,2,3,3a,4,8b-hexahydro-2-benzyl-6-N,N-dimethylamino-1-methylindeno[1,2,-b]pyrrole), and perfloxacin, and/or analogs or metabolites thereof, or pharmaceutical salts thereof.

In some embodiments, the subject has been exposed to binge or binge-like drinking in adolescence with or without family history of drinking.

### Kits

In a further aspect, kits are provided by the present disclosure, such kits comprising: one or more pharmaceutical compositions, each composition sterilized within a container, means for administration of the pharmaceutical compositions to a subject, and instructions for use.

Some embodiments include kits for carrying out the methods disclosed herein. Such kits typically comprise two or more components required for treating a clinical condition. Components of the kit include, but are not limited to, one or more of agents/compositions disclosed herein, reagents, containers, equipment and/or instructions for using the kit. Accordingly, the compositions and methods described herein can be performed by utilizing pre-packaged kits disclosed herein.

### EXAMPLES

The following examples illustrate various aspects of the disclosure. It will be apparent to those skilled in the art that many modifications, both to materials and methods, can be practiced without departing from the scope of the disclosure.

Subjects. The alcohol-preferring (P) rats used in the current experiments were from the standard P stock maintained by the Animal Breeding Core of the Indiana University Alcohol Research Center (IUARC). Animals used in this study were maintained in facilities fully accredited by the Association for the Assessment and Accreditation of Laboratory Animal Care. All research protocols were approved by the IUSM Institutional Animal Care and Use Committee (IUSM IACUC) and were in accordance with the guidelines of the Institutional Care and Use Committee of the National Institute on Drug Abuse, the NIH, and the Guide for the Care and Use of Laboratory Animals (2011).

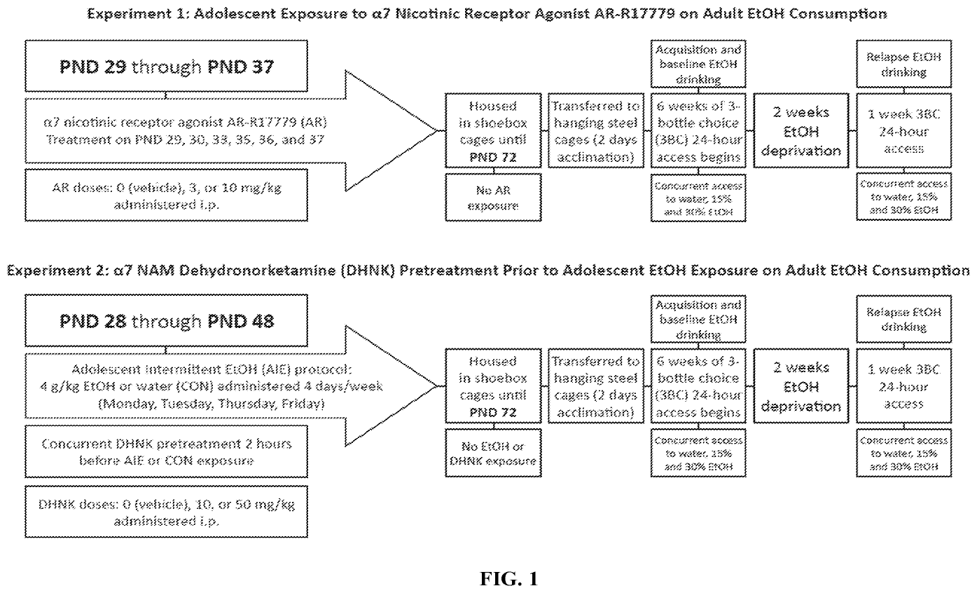

Effects of exposure to the α7 agonist AR-R17779 during early adolescence on adult EtOH consumption. Adolescent male (n=14) and female (n=13) alcohol preferring (P) rats were exposed to the α7 nicotinic receptor agonist, AR-R17779 ((2S)-2′H-spiro[4-azabicyclo[2.2.2]octane-2,5′-[1,3]oxazolidin]-2′-one) (AR; Tocris) on PND 29, 30, 33, 35, 36, and 37. The doses of AR-R17779 was 0 (saline controls), 3 or 10 mg/kg (all drugs administered i.p.). AR is a highly selective α7 nicotinic receptor agonist with no or minimal affinity for all other examined receptors. The doses of AR employed are typical for peripheral administered studies and results in observed alterations in specific behaviors. All rats were group housed between PND 26 and 73 (adulthood). Subjects only received treatment with AR_on the days specified. Experiment Timeline is provided in FIG. 1 (top panel).

Statistical Analysis. All statistical analyses followed the procedures/flowchart indicated by Keppel and Zedeck (1986). Briefly, the experiments examining the acquisition of alcohol consumption utilized the dependent measure of average weekly alcohol intake. The overall analyses were mixed factor ANOVAs with between subject factors of drug dose and EtOH exposure history (when applicable) and a within subject factor of Week. The statistical analyses surveying relapse drinking examined the average EtOH intake for the three days prior to deprivation and daily EtOH intake values following re-exposure. Post-hoc comparisons for between subject factors were performed with Tukey's b analyses. The Tukey's b post-hoc comparison is a modified Tukey post-hoc comparison that reduces the prohibitive penalty for unequal sample size. There is a single assumption of ANOVAs that cannot be violated. The statistical integrity of ANOVAs is greatly reduced when the assumption of ‘Independence of Measure’ is violated (Kepple and Zeddeck, 1986). Therefore, within subject variables should not be treated like between subject variables and analyzed with forced ANOVAs and inappropriate post-hoc analyses. Proper within subject post-hoc analyses are t-tests and orthogonal contrasts (Keppel and Zeddeck, 1986). Given the general lack of understanding of orthogonal contrasts, the findings from t-test analyses are being reported. Concerns of Type I error rate inflation is eliminated by the proper reading of Rodgers (1967). Type 1 error rate inflations is Ψᵢ (i=1, . . . , J−1), and not Bonferroni, an improper estimate. Given the effect size of the current data set there is no likelihood of Type 1 error rate inflation for any reported analyses (c.f., Rodgers and Roberts, 2012; Roberts, 2011).

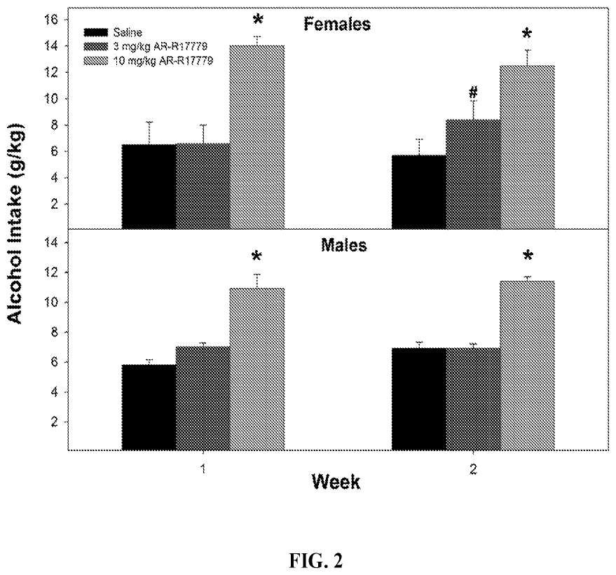

Referring now to FIG. 2, statistical analysis performed on the dependent measure of average daily alcohol intake for each week revealed a Week×Sex×Dose interaction (F_{2,21}=4.59; p=0.022). For both weeks, there was a significant effect of AR dose on alcohol intake (F_{2,21} values>24.58; p<0.0001). In female rats (top panel) there was a significant effect of AR on adult alcohol consumption for the first two weeks of alcohol drinking (F_{2,10} values>9.6; p<0.005). Post-hoc comparisons (Tukey's b) indicated that during the 1^{st} week of alcohol access female P rats administered 10 mg/kg AR during early adolescence consumed more alcohol than rats administered saline or 3 mg/kg AR during adolescence. During the 2^{nd} week of alcohol access, post-hoc comparisons indicated that rats treated with 10 mg/kg AR during early adolescence consumed more alcohol than rats treated with 3 mg/kg AR which consumed more than saline treated rats (10 mg/kg>3 mg/kg>saline; p<0.05). In male rats (bottom panel) there was a significant effect of AR on adult alcohol consumption during weeks 1 and 2 (F_{2,11}=21.1; p<0.001). Post-hoc comparisons indicated that during the 1^{st} and 2^{nd} weeks of alcohol access, male rats administered 10 mg/kg AR during early adolescence consumed more alcohol than rats administered saline or 3 mg/kg AR (p<0.05). Baseline alcohol consumption was determined by average intake during the last 3 days prior to deprivation. Overall analysis indicated a significant Day×Dose×Sex interaction term (FIG. 2; F_{8,84}=3.18; p=0.003). Reducing the interaction term by examining effects as a factor of Sex was performed utilizing established statistical procedures.

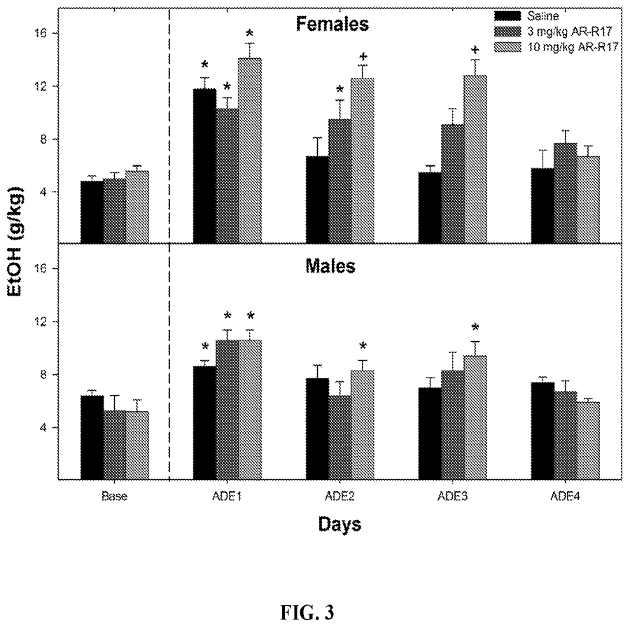

Referring now to FIG. 3, in female rats (top panel) there was a significant Day×Dose interaction term (F_{8,40}=5.9; p<0.001). There was no group difference in the amount of EtOH consumed during the last 3 days of baseline intake (p=0.624). Individual ANOVAs indicated that during re-exposure days 1-3 there were significant group differences (F_{2,10} values>4.98; p<0.031). Post-hoc comparisons (Tukey's b; p<0.05) revealed that on Day 1 of EtOH re-exposure the group of rats treated with 10 mg/kg AR consumed more alcohol than the 3 mg/kg group. During the 2^{nd} re-exposure day, rats administered 10 mg/kg AR consumed more alcohol than rats administered saline. Each group differed significantly from each other on re-exposure day 3 (10 mg/kg>3 mg/kg>Saline). Within subject analysis indicated that rats administered Saline during adolescence displayed an increase in EtOH consumption during the 1^{st} re-exposure day compared to baseline level intake. Evidence for an ADE was observed in rats treated with 3 mg/kg (2 days) and 10 mg/kg (3 days).

The expression of an ADE was reduced in males compared to females (FIG. 3, bottom panel). Statistically, there was a Day×Dose interaction (F_{2,11}=2.67; p=0.017). There were no significant group differences between the AR male groups during re-exposure days 1-4 (p>0.083). Within subject analysis indicated that male rats administered Saline or 3 mg/kg AR during adolescence displayed elevated levels of EtOH intake during the 1^{st} re-exposure day compared to baseline intake (p<0.05). Evidence of an ADE was detected during re-exposure days 1-3 in rats administered 10 mg/kg AR during adolescence.

Effects of exposure to the α7 agonist AR-R17779 during early adolescence on adult EtOH consumption (acquisition, maintenance, extinction, craving, and relapse). The current experiment was conducted to generalize the effects of exposure to an α7 NAchR receptor agonist during adolescence on adult alcohol consumption in non-selected (non-genetically predisposed rats). Brief exposure to AR during adolescence in Wistar rats produced similar alterations in adult beer consumption. Male and female Wistar rats were exposed to AR (0, 3, or 10 mg/kg) on PND 29, 30, 33, 35, 36, and 37 (no alcohol exposure during adolescence). On PND 75 rats were allowed to self-administer beer (9% IPA) under operant conditions.

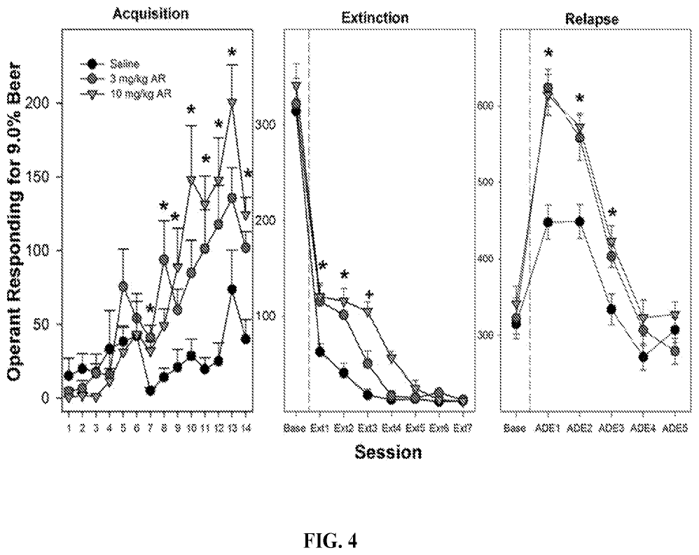

Referring now to FIG. 4, brief adolescent AR exposure in Wistar rats produced similar effects of alcohol consumption during adolescence (e.g., enhanced acquisition, retardation of extinction, and enhanced relapse drinking). Acquisition of alcohol self-administration tests the intrinsic rewarding properties of a drug. Statistically, there was a significant Session X AR concentration during the initial 14 days of adult alcohol access (F_{26.498}=2.1; p<0.001). Post-hoc comparisons indicated from session 7-14, rats exposed to the α7 NAchR agonist AR-R17779 during adolescence acquired alcohol self-administration faster than controls (F_{2,23} values>12.59; p values<0.022). Extinction training (FIG. 4; middle panel) tests an animal's willingness to stop responding for a previous reward. The rate of extinction indicates an organism's; 1) tolerance to the frustration of losing a reward, 2) perseveration on obtaining a ‘lost’ reward, and 3) the strength of drug-paired environments to stimulate self-administration behaviors. Baseline responding (average of the last three sessions with alcohol available prior to extinction training) for all groups is indicated as Base in the figure (middle panel). All experiment groups were self-administering equivalent amounts of alcohol prior to extinction training (F_{2,23}=0.68; p=0.87). Statistical analysis indicated that adolescent exposure to the α7 NAchR agonist AR-R17779 retarded the rate of extinction learning. Individual ANOVAs performed on each extinction sessions indicated significant group differences during the first 4 extinction sessions (F_{2,23} values>12.97; p values<0.001). Post-hoc comparisons (Tukey's b) indicated that during extinction sessions 1 and 2, Wistar rats exposed to the α7 NAchR agonist AR-R17779 during adolescence responded more on the lever previously associated with the delivery of alcohol (no alcohol available during extinction training) than saline treated rats. During extinction session 3, groups were significantly different from each other. Adolescent Wistar rats exposed to the highest dose of the α7 NAchR agonist AR-R17779 responded more on the lever previously associated with delivery of alcohol than all other groups. Adolescent exposure to the α7 NAchR agonist AR-R17779 also promoted relapse drinking behavior in Wistar rats. Statistically, there was a significant Session X AR dose during alcohol re-exposure (F_{8,144}=3.37; p<0.001). Preclinical relapse drinking is considered to emulate the human phenomenon of individuals consuming more alcohol following a period of abstinence (alcohol deprivation effect) than under ‘normal’ drinking conditions. While all groups responded more during the initial re-exposure period, individual ANOVAs indicated that adolescent exposure to the α7 NAchR agonist AR-R17779 enhanced relapse drinking (F_{2,23} values>14.25; p values<0.001). Post-hoc comparisons indicated that rats exposed to the α7 NAchR agonist AR-R17779 during adolescence consumed more alcohol than control rats (saline) during the initial 3 re-exposure sessions. Overall, the data indicate that exposure to an the α7 NAchR agonist during adolescence enhances alcohol consumption during adulthood under multiple measures.

Effects of exposure to the α7 negative allosteric modulator (NAM) Dehydronorketamine prior to adolescence binge alcohol exposure on adult EtOH consumption. Dehydronorketamine (DHNK) is a metabolite of ketamine but is lacking glutamatergic effects. DHNK has no reported abuse liability and is a selective α7 NAM (binding to α3β4 IC₅₀>200 μM). A NAM does not reduce the activity of a receptor; NAMs prevents the increase or decrease activity of a receptor produced by ligands. The DI-INK concentrations used in the reported studies were comparable to concentrations of DHNK shown not to influence locomotor activity or other variables. The use of an α7 nicotinic receptor NAM is beneficial to use in adolescent subjects because it does not alter the normal activity of a receptor.

Effects of pretreatment with an α7 NAM dehydronorketamine prior to binge-like exposure during adolescence on adult EtOH consumption. There were 5 treatment groups. Adolescent male (n=24) and female (n=26) P rats were used in the experiment. The AIE exposure began on PND 28. Rats were given 4 g/kg alcohol (25% v/v; gavage) every Monday, Tuesday, Thursday and Friday (4 days a week; 3 days with no alcohol exposure) from post-natal day 28-48. Control (CON) groups received an equivalent volume administration of water. In the AIE groups, rats were injected (i.p.) with 0 (saline), 10 or 50 mg/kg of the α7 nicotinic receptor NAM dehydronorketamine (DHNK; Santa Cruz biotechnology) 2 hours prior to AIE exposure. CON groups received (i.p.) injections of saline or 50 mg/kg DHNK 2 hours prior to water gavage exposure. All rats were group housed between post-natal day 48 and 73 (adulthood). Pretreatment and gavage administration occurred during PND 28, 29, 30, 31, 34, 35, 37, 39, 40, 41, 44, 45, 47, and 48. Subjects were not exposed to DHNK any time after PND48. Experiment Timeline is provided in FIG. 1 (bottom panel).

There were no differences observed between ATE and CON rats for body weight during adolescence (data not shown), no loss of subjects, no signs of stress during the procedure, and no observed behavioral effects of gavage.

Statistical analysis performed on the dependent measure of average daily alcohol intake for each week revealed a Week×Dose interaction (F_{4,40}=3.42; p=0.017). Overall, pretreatment with DHNK blocked the ability of adolescent alcohol exposure to increase alcohol consumption during adulthood (FIG. 4).

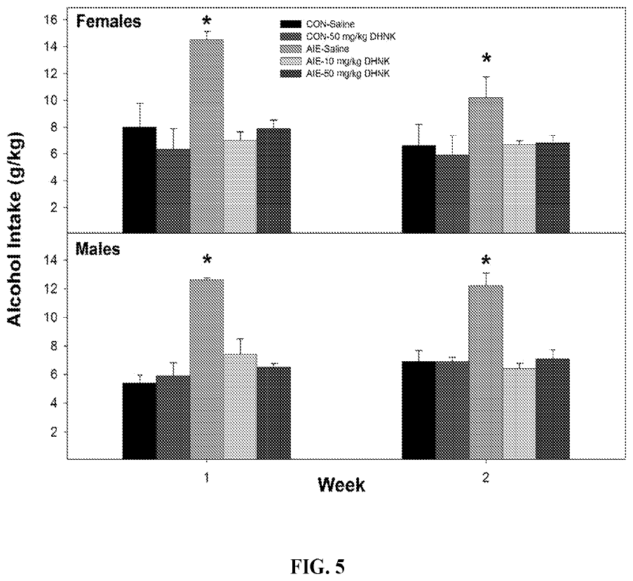

Referring now to FIG. 5, in female rats (top panel) there was a significant effect of DHNK dose during adolescence on the average weekly alcohol intake in adulthood during the first and second weeks of alcohol access (F_{4,21}=3.42; p<0.027). Post-hoc comparisons (Tukey's b) indicated that rats exposed to the AIE protocol drank significantly more alcohol during the 1^{st} and 2^{nd} week of alcohol access compared to all other groups. In male rats (FIG. 5, bottom panel) there was a significant effect of DHNK administration during adolescence on the ability of the AIE treatment to enhance alcohol drinking during adulthood (F_{4,19} values>8.91; p<0.001). Post-hoc comparisons indicated that during the 1^{st} and 2^{nd} week of alcohol access, male rats pretreated with saline prior to AIE exposure consumed more alcohol than all other groups. The data indicated that 1) exposure to the high dose of DHNK did not alter alcohol drinking during adulthood (CON-50 mg/kg DHNK), indicating a lack of abnormal developmental actions, 2) adolescent alcohol exposure increased adult alcohol consumption in both males and females (AIE-Saline), and 3) pretreatment with 10 or 50 mg/kg DHNK blocked the deleterious effects of adolescent alcohol exposure (FIGS. 2-4).

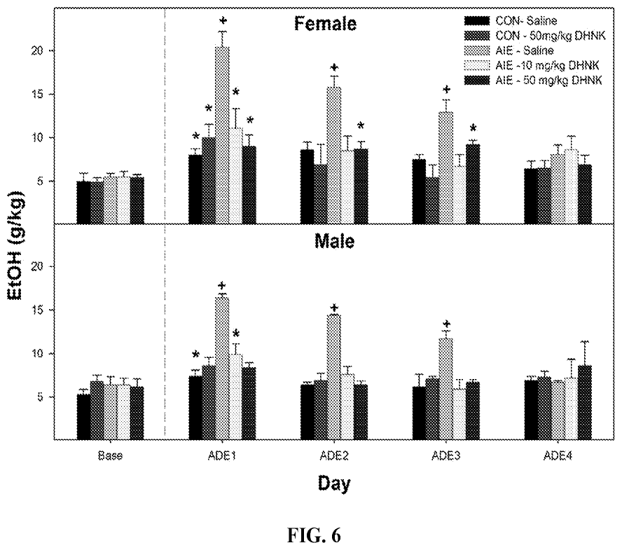

Referring now to FIG. 6, DHNK pretreatment altered the enhancement in relapse drinking produced by AIE exposure. The overall analysis indicated significant Day×Sex (F_{4,160}=3.44; p<0.001) and Day×Dose (F_{4,160}=7.25; p<0.0001), but no Sex×Day×Dose interaction (p=0.96). Given a priori considerations, the interaction term were decomposed by examining Day and Dose in each sex. In female rats (FIG. 6, top panel) there was a significant Day×Dose interaction term (F_{4,76}=4.3; p<0.001). There was no group difference during baseline intake (F_{4,21}=0.104; p=0.98). During the first three re-exposure days there were significant group differences in alcohol intake (F_{4,21} values>5.28; p<0.004). Post-hoc comparisons (Tukey's b) indicated that the AIE-Saline group consumed more alcohol than all other groups.

Given that this analysis was a within subject procedure, paired t-tests were performed to determine within group alterations from baseline consumption. Adolescent rats given control water gavage exposure (CON-Saline and CON-50 mg/kg DHNK) displayed a significant increase in alcohol drinking during the 1^{st} re-exposure day. Adolescent EtOH exposure potentiated alcohol relapse drinking. The AIE-Saline and ATE-50 mg/kg DHNK expressed an increase in alcohol consumption during the first three re-exposure days. Rats in the AIE-10 mg/kg DHNK expressed an increase in alcohol consumption (compared to baseline during the 1^{st} re-exposure day).

In male rats (FIG. 6, bottom panel) there were a similar Day×Dose interaction term (F_{16, 76}=3.8; p<0.001). There were no group differences in baseline EtOH consumption (p=0.71). There were significant group differences during re-exposure days 1-3 (F_{4, 19} values>4.03; p<0.016). Post-hoc comparisons (Tukey's b) indicated that the AIE-Saline group consumed more alcohol than all other groups during re-exposure days 1-3. Within subject analysis indicated that there was a significant increase in alcohol consumption from baseline intake during re-exposure days 1-3 in the AIE-Saline group. Rats in the CON-Saline and AIE-10 mg/kg DHNK groups displayed increase in drinking during the 1^{st} re-exposure day. Rats in the CON-50 mg/kg and AIE-50 mg/kg groups did not display an ADE.

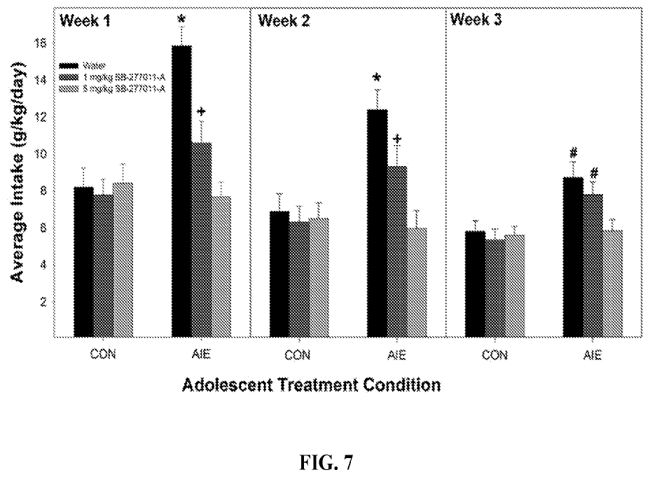

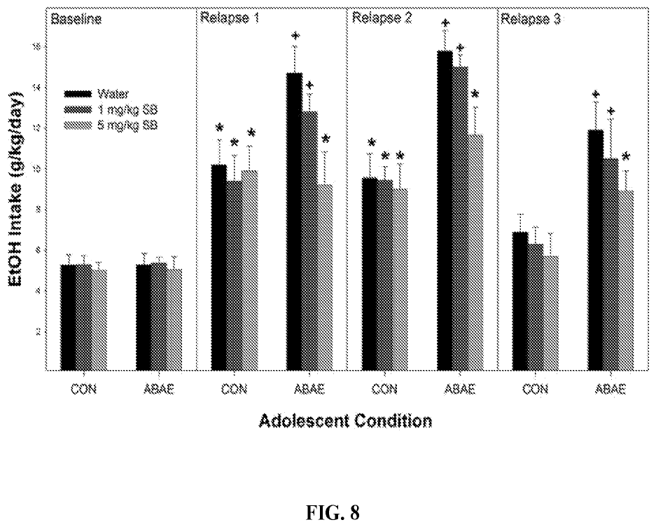

Effects of exposure to the α7 negative allosteric modulator (NAM) SB-277011-A prior to adolescence binge alcohol exposure on adult EtOH consumption. Currently, there are 3 commercially available α7 NAMs: DHNK, SB-277011-A (SB), and Butaclamol (AY-23,028). SB is also a selective D3 antagonist. Butaclamol is a typical antipsychotic that is also a non-selective dopamine receptor antagonist. Referring now to FIG. 7, the ability of SB to prevent ABAE-induced increases were assessed in adult EtOH consumption. Briefly, female P rats were exposed to the standard ABAE protocol. ABAE and CON rats were injected (i.p.) with 0 (water), 1 or 5 mg/kg SB (Sigma) 2 hours before gavage treatment. Similar to DHNK, pretreatment with SB prevented the ABAE-induced increase in EtOH consumption during acquisition in a dose—there was a significant ABAE×SB Dose×Week interaction term (F_{4,147}=3.68; p<0.001). Similar to the DHNK data, pretreatment with SB before ABAE administration (gavage) prevented ABAE-induced enhancement of EtOH consumption during relapse (ADE) conditions (FIG. 8). Statistically, there was a significant ABAE X SB Dose X Day interaction term (F_{12,244}=2.3; p<0.01). Therefore, the data sets indicate that despite having different off-target sites of activation (SB-D3 antagonist), both α7 NAMs prevented the deleterious consequences of ABAE.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety to the extent they are not inconsistent with the explicit teachings of this specification.

1. A method of treating an alcohol use disorder or a related condition thereof, comprising:
administering to a subject at least one therapeutically effective dose of at least one agent comprising at least one negative modulator of alpha-7 nicotinic receptor.

2. The method of claim 1, wherein the at least one negative modulator comprises at least one of: norketamine ((2-amino-2-(2-chlorophenyl)cyclohexan-1-one), dehydronorketamine (5,6-dehydronorketamine), hydroxynorketamine ((2-amino-2-(2-chlorophenyl)-6-hydroxycyclohexan-1-one), SB-27701-A ((N-{trans-4-[2-(6-cyano-3,4-dihydroisoquinolin-2(1H)-yl)ethyl]cyclohexyl}quinoline-4-carboxamide), butaclamol ((3 S,4aS,13b S)-3-(2-methyl-2-propanyl)-2,3,4,4a,8,9,13b,14-octahydro-1Hbenzo[6,7]cyclohepta[1,2,3-de]pyrido[2,1-a]isoquinolin-3-ol), DB04763 ((2,4,6-trimethyl-1-[2-(4-sulfamoylphenyl)ethyl]pyridin-1-ium), DB08122 ((N-methyl-4-({[(3Z)-2-oxo-2,3-dihydro-1H-indol-3-ylidene]methyl}amino)benzene-1-sulfonamide), MD-354 (2-(3-chlorophenyl)-1-(diaminomethylidene)guanidine), HDMP ((1,2,3,3a,4,8b-hexahydro-2-benzyl-6-N,N-dimethylamino-1-methylindeno[1,2,-b]pyrrole), and perfloxacin, and/or analogs or metabolites thereof, or pharmaceutical salts thereof.

3. The method of claim 2, wherein the at least one negative modulator comprises at least one of: dehydronorketamine (5,6-dehydronorketamine), SB-27701-A ((N-{trans-4-[2-(6-cyano-3,4-dihydroisoquinolin-2(1H)-yl)ethyl]cyclohexyl}quinoline-4-carboxamide), and butaclamol ((3S,4aS,13b S)-3-(2-methyl-2-propanyl)-2,3,4,4a,8,9,13b,14-octahydro-1Hbenzo[6,7]cyclohepta[1,2,3-de]pyrido[2,1-a]isoquinolin-3-ol), and/or analogs or metabolites thereof, or pharmaceutical salts thereof.

4. The method of any one of claim 1, wherein the subject is diagnosed with or at risk of having an alcohol use disorder, a drug or alcohol addiction, an anxiety disorder, or a related condition thereof.

5. The method of any one of claims, wherein the subject is diagnosed with or at risk of having or has increased likelihood of developing schizophrenia, Alzheimer's disease, Parkinson's disorder, other dementia-related illnesses, Type II diabetes, and autoimmune diseases.

6. The method of any one of claim 1, wherein the subject is an adolescent or a young adult in the age between 9 to 25 years old.

7. The method of any one of claim 1, wherein the therapeutically effective dose of at least one agent comprises from about 0.01 mg to about 1000 mg, from about 0.01 mg to about 500 mg, from about 0.01 mg to about 200 mg, from about 0.01 mg to about 100 mg, from about 0.01 mg to about 50 mg, from about 0.01 mg to about 10 mg, from about 0.1 mg to about 100 mg, from about 1 mg to about 100 mg, and/or from about 10 mg to about 500 mg.

8. The method of any one of claim 1, further comprising the step of:
identifying a subject who had experienced binge drinking.

9. The method of any one of claim 1, further comprising the step of:
administering to the subject the therapeutically effective dose of at least one agent at least once prior to, during, or subsequent to alcohol consumption.

10. The method of any one of claim 1, wherein the therapeutically effective dose of at least one agent is administered to the subject at least once within four hours prior to or subsequent to alcohol consumption.

11. A method of reducing alcohol consumption, comprising:
administering to an adolescent subject at least one therapeutically effective dose of at least one agent comprising at least one negative modulator of alpha-7 nicotinic receptor.

12. The method of claim 11, wherein the at least one negative modulator comprises at least one of: norketamine ((2-amino-2-(2-chlorophenyl)cyclohexan-1-one), dehydronorketamine (5,6-dehydronorketamine), hydroxynorketamine ((2-amino-2-(2-chlorophenyl)-6-hydroxycyclohexan-1-one), SB-27701-A ((N-{trans-4-[2-(6-cyano-3,4-dihydroisoquinolin-2(1H)-yl)ethyl]cyclohexyl}quinoline-4-carboxamide), butaclamol ((3 S,4aS,13b S)-3-(2-methyl-2-propanyl)-2,3,4,4a,8,9,13b,14-octahydro-1Hbenzo[6,7]cyclohepta[1,2,3-de]pyrido[2,1-a]isoquinolin-3-ol), DB04763 ((2,4,6-trimethyl-1-[2-(4-sulfamoylphenyl)ethyl]pyridin-1-ium), DB08122 ((N-methyl-4-({[(3Z)-2-oxo-2,3-dihydro-1H-indol-3-ylidene]methyl}amino)benzene-1-sulfonamide), MD-354 (2-(3-chlorophenyl)-1-(diaminomethylidene)guanidine), HDMP ((1,2,3,3a,4,8b-hexahydro-2-benzyl-6-N,N-dimethylamino-1-methylindeno[1,2,-b]pyrrole), and perfloxacin, and/or analogs or metabolites thereof, or pharmaceutical salts thereof.

13. The method of claim 12, wherein the at least one negative modulator comprises at least one of: dehydronorketamine (5,6-dehydronorketamine), SB-27701-A ((N-{trans-4-[2-(6-cyano-3,4-dihydroisoquinolin-2(1H)-yl)ethyl]cyclohexyl}quinoline-4-carboxamide), and butaclamol ((3S,4aS,13bS)-3-(2-methyl-2-propanyl)-2,3,4,4a,8,9,13b,14-octahydro-1Hbenzo[6,7]cyclohepta[1,2,3-de]pyrido[2,1-a]isoquinolin-3-ol), and/or analogs or metabolites thereof, or pharmaceutical salts thereof.

14. The method of any one of claim 11, wherein the adolescent subject has been exposed to binge or binge-like alcohol consumption.

15. The method of any one of claim 11, wherein the adolescent subject is diagnosed with or at risk of having or has increased likelihood of developing alcohol use disorder, a drug or alcohol addiction, an anxiety disorder, schizophrenia, Alzheimer's disease, Parkinson's disorder, other dementia-related illnesses, Type II diabetes, and autoimmune diseases, or a related condition thereof when the adolescent subject becomes an adult.

16. The method of any one of claim 11, wherein the adolescent subject is in the age between 9 to 20 years old.

17. The method of any one of claim 11, wherein the therapeutically effective dose of at least one agent comprises from about 0.01 mg to about 1000 mg, from about 0.01 mg to about 500 mg, from about 0.01 mg to about 200 mg, from about 0.01 mg to about 100 mg, from about 0.01 mg to about 50 mg, from about 0.01 mg to about 10 mg, from about 0.1 mg to about 100 mg, from about 1 mg to about 100 mg, and/or from about 10 mg to about 500 mg.

18. The method of any one of claim 11, further comprising the step of:
administering to the adolescent subject the therapeutically effective dose of at least one agent at least once prior to, during, or subsequent to alcohol consumption.

19. The method of any one of claim 11, further comprising the step of:
administering to the adolescent subject the therapeutically effective dose of at least one agent at least once prior to binge or binge-like alcohol consumption.

20. The method of any one of claim 1, wherein the therapeutically effective dose of at least one agent is administered to the subject at least once within four hours prior to or subsequent to binge or binge-like alcohol consumption.
